(19) United States
(12) Patent Application Publication
(10) Pub. No.: US 2026/0031220 A1
(43) Pub. Date: Jan. 29, 2026

(54) SCALE CONTROLLERS FOR AI-BASED SUPPLY MANAGEMENT

(71) Applicant: PAR Excellence Systems, Inc., Cincinnati, OH (US)

(72) Inventors: Thaddeus MacKrell, Grosse Pointe City, MI (US); Thomas Joseph Kochan, II, Brighton, MI (US)

(21) Appl. No.: 19/278,331

(22) Filed: Jul. 23, 2025

Related U.S. Application Data
(60) Provisional application No. 63/674,580, filed on Jul. 23, 2024.

(51) Int. Cl. G16H 40/20; G01G 19/414; G06Q 10/087

(52) U.S. Cl. CPC G16H 40/20; G01G 19/414; G06Q 10/08726

(57) ABSTRACT

Methods and systems are described for scale-based inventory management and monitoring at a location, such as hospitals. Scales may be configured to receive a tray/holder for a given type of supply item, e.g., bandages or syringes. A weight determined by the scale may be associated with a given quantity of the respective item. Data around supply delivery and restocking is collected and can be used in an AI/ML model to optimize delivery routes, labor options for delivery, delivery speed, or other factors useful in optimizing supply and logistics in any location with logistics challenges.

### CROSS REFERENCE TO RELATED INFORMATION

This application claims the benefit of United States of America priority application No. 63/674,580, filed on Jul. 23, 2024, titled “Scale Controllers for AI-Based Supply Management,” the contents of which are hereby incorporated herein in its entirety.

### TECHNICAL FIELD

The present disclosure generally relates to systems and methods for supply item monitoring.

### BACKGROUND

In a variety of physical locations, medical supplies must be able to be stored and accessed. For example, nursing facilities, medical offices, and hospitals, among others, have ongoing needs for supplies having medical-related applications. For example, in a hospital having operating room capabilities, sutures are regularly required by the surgeon. Depending upon the nature of the surgery, a variety of sutures must be readily available for use by the surgeon undertaking the medical procedure. Further, a still more specialized grouping of sutures can be required in an individual operating arena, to be immediately available during the course of a surgery. Thus, sutures used in performing joint replacements will be of a different size and composition than those used in delicate neurosurgery. As a result, though a central core storage area in an operating room center may contain a wide range of sutures, in the individual operating room a much more specific suture selection is needed.

Some prior art has attempted to solve some or similar issues, but they all have drawbacks.

For example, the Central Carolina Scale is a weight transmitter made by Central Carolina Scale. But it is only a weight transmitter (not a scale or controller by itself). Another prior art system is the MT-2024 ACT Weight Transmitter. This is a web enabled PLC (Programmable Logic Controller). Another prior art reference is the CardinalScale. This is a measurement tool similar to the Central Carolina Scale, with similar drawbacks.

Another example of prior art is ControlbyWeb. This is a X-400 web enabled PLC (like the MT-2024 Act Weight Transmitter).

Another prior art reference is United States Publication No. 2021/0295248, “System and Method for Inventory Management” by Venture Measurement Company, LLC. This inventory management system is designed for use for tracking the weight of raw material (grains, pellets, etc.). Another reference is United States Publication No. 2023/0260631, for “Inventory System, Devices and Methods Thereof” by MedVision AI Corp. This is an inventory management system for medical environments. It tracks inventory on shelves using “weight sensors” but is shelf-based, not louvre or any other type. Also, it appears to be platform type only and does not have WiFi and Cellular network options. Another reference is United States Publication No. 2008/0082360, for “Inventory Monitoring and Control Application” by Clear-View-Technologies, Inc. This is a device for measuring dispensed product for the prevention of theft (i.e., pharmaceuticals). It is reliant on a local computer and the scale is tared between dispensed items, and only has a single point of use.

Another reference is U.S. Pat. No. 10,332,066, for “Item management system using weight” by Amazon Technologies, Inc. This is an inventory management process using scales, RFID tags, etc. This is less about the network, scales and more about the management of the data once it has been gathered. For example, multi-factor can be used to determine if weight/count is stable, helping to determine if inventory actually changed or “if a train is going by.”

Another reference is United States Publication No. 2022/0113182, for “Communicating Weight Sensor Units and Techniques For Using Same” by Inventory-E Limited. This is a wireless inventory management system, but is shelf or table based only-no louver options and is targeted to production/assembly lines. Another reference is U.S. Pat. No. 10,275,740, for “Consumable usage sensors and applications to facilitate automated replenishment of consumables via an adaptive distribution platform” by OrderGroove, Inc. This is a control system to manage inventory using “various sensors” but revolves around the system backend more than on how their data is acquired. Another reference is U.S. Pat. No. 11,087,603, for “Inventory management system and wireless tag device” by Zensho Holdings Co., LTD. This is an inventory management system using an RF tag to transmit weight-based data. This is an RFID type of scale and is shelf-based only, designed for retail (restaurant) environments, and battery powered.

Another reference is U.S. Pat. No. 6,639,156, for “Method and device monitoring inventory,” by NBH Capital Finance, A Division of NBH Bank, N.A. This is for an electronic inventory system using modular load cell interface. But in this reference load cells are combined per A/D converter and there is no indication about how these are networked together.

Other references include United States Publication No. 2017/0148005, for “Integrated Automatic Retail System and Method” by Via Touch Media, Inc.; U.S. Pat. No. 11,713,995, for “System and method for managing inventory,” by Diebold Nixdorf Systems GMBH; U.S. Pat. No. 11,125,607, for “Integrated multi-lane weight measuring device” by Amazon Technologies, Inc.; United States Publication No. 2022/0222616, for “Sensor Based Item Level Determination and Communication” by Ypoint Capital, Inc.; and Rice Lake-2024 a weight indicator and controller. These references have many of the same drawbacks and disadvantages as the references described above.

### SUMMARY

One embodiment under the present disclosure comprises a system for monitoring a supply inventory. The system comprises: a first scale comprising a first load cell and configured to receive a first tray holder, the first tray holder configured to retain a first supply item, wherein the first load cell is configured to detect a first weight of the first tray holder. The system also comprises a first scale controller comprising a first processor and a first memory, the first scale controller configured to receive the first weight from the first scale and to associate the first weight to a quantity of the first supply item, the first scale controller configured to determine a status of the first supply item based on the first weight of the first tray holder; and a first remote computing device communicatively coupled to the first scale controller and configured to monitor a status of the first supply item, wherein the first scale or the first scale controller is operable to perform training of a machine learning model for optimizing supply delivery, the training comprising: obtaining a dataset of identified supply-related outcomes; training the machine learning model using the dataset of identified supply-related outcomes thereby obtaining a trained machine learning model; and storing the trained machine learning model.

Another embodiment under the present disclosure comprises a system for monitoring an inventory at a location. The system comprises: a first scale comprising a first load cell and configured to receive a first tray holder, the first tray holder configured to retain a first supply item, wherein the first load cell is configured to detect a first weight of the first tray holder; a second scale comprising a second load cell and configured to receive a second tray holder, the second tray holder configured to retain a second supply item, wherein the second load cell is configured to detect a second weight of the second tray holder; and a first scale controller, the first scale controller configured to receive the first weight from the first scale and to associate the first weight to a quantity of the first supply item, the first scale controller configured to determine a status of the first supply item based on the first weight of the first tray holder, the first scale controller further configured to receive the second weight from the second scale and to associate the second weight to a quantity of the second supply item, the first scale controller configured to determine a status of the second supply item based on the second weight of the second tray holder. The system further comprises: a first mobile cart configured to carry the first and second supply items, the first mobile cart comprising a first scanner configured to scan the first and second supply items and record a presence of the first and second supply items on the first mobile cart; a first sensor at the location, the first sensor operable to detect a location of the first and second supply items. The system also includes: a first server communicatively coupled to the first scale controller, to the first mobile cart, and the first sensor, and configured to monitor the status of the first supply item, the status of the second supply item, the location of the first and second supply items, and the presence of the first and second supply items on the first mobile cart, wherein the first scale or the second scale or the first scale controller is operable to perform training of a machine learning model for optimizing supply delivery, the training comprising: obtaining a dataset of identified supply-related outcomes; training the machine learning model using the dataset of identified supply-related outcomes thereby obtaining a trained machine learning model, and storing the trained machine learning model.

A further embodiment under the present disclosure comprises a computer implemented method for training a machine learning model for optimizing supply delivery. The method comprises obtaining a dataset of identified supply-related outcomes; training the machine learning model using the dataset of identified supply-related outcomes thereby obtaining a trained machine learning model and storing the trained machine learning model.

A further embodiment comprises a method of monitoring supply items. The method includes: receiving a first tray holder in a first scale, the first tray holder configured to retain a first supply item; detecting, by a first load cell in the first scale, a weight of the first tray holder; and transmitting the weight of the first tray holder to a first scale controller. The method further comprises: associating, by the first scale controller, the weight of the first tray holder to an associated quantity of the first supply item; determining, by the first scale controller, a status of the first supply item based on the associated quantity; and transmitting, by the first scale controller, the status of the first supply item to a first remote computing device configured to monitor the status of the first supply item.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

For a more complete understanding of the present disclosure, reference is now made to the following descriptions taken in conjunction with the accompanying drawings, in which:

FIG. 1 illustrates one embodiment of an inventory monitoring system under the present disclosure;

FIG. 2 shows an example embodiment of a supply cabinet under the present disclosure;

FIG. 3 shows an example embodiment of tray holders under the present disclosure;

FIG. 4 shows an example embodiment of an sensor/scale under the present disclosure;

FIG. 5 shows an example embodiment of a scale controller under the present disclosure;

FIGS. 6A-6C show an example embodiment of a scale controller under the present disclosure;

FIGS. 7A-7B show an example embodiment of a scale controller under the present disclosure;

FIG. 8 shows an example embodiment of a scale controller under the present disclosure;

FIG. 9 shows an embodiment of a computing device for use in various embodiments under the present disclosure;

FIG. 10 shows an example flow chart of training and inference pipelines for machine learning in accord with some embodiments under the present disclosure;

FIG. 11 shows an embodiment of a neural network under the present disclosure;

FIG. 12 illustrates a flow-chart of a method embodiment under the present disclosure; and

FIG. 13 illustrates a flow-chart of a method embodiment under the present disclosure.

### DETAILED DESCRIPTION

Before describing various embodiments of the present disclosure in detail, it is to be understood that this disclosure is not limited to the parameters of the particularly exemplified systems, methods, apparatus, products, processes, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed embodiments. In addition, the terminology used herein is for the purpose of describing the embodiments and is not necessarily intended to limit the scope of the claimed embodiments.

There currently exist certain challenges in the realm of medical supply inventory management, tracking, and logistics. Current solutions are often weight transmitters, but with little local controller functionality, or the transmitter has the inability to detect precise weights. Some systems are shelf-based, not usable with louvers or other mechanisms that allow for greater detection ability. Other systems do not network together well, allowing for only a single point of use. Some systems have backend data analysis capability, but little ability to analyze or control components or data locally. Current systems also lack the ability to predict or optimize supply-related tasks for outcomes.

Certain aspects of the embodiments disclosed herein provide solutions to these or other challenges. Embodiments include a CAN (controller area network) bus scale controller that can be coupled to, and provide communication services to, a plurality of scales in e.g., a hospital storeroom or other inventory system. Disclosed systems also bring artificial intelligence and machine learning capabilities for optimizing various supply related outcomes, such as labor costs, supply costs, delivery speed, or other outputs.

Certain embodiments may provide one or more of the following technical advantages over the prior art. Existing scale/inventory monitoring solutions may be coupled to a plurality of devices, but perform a round robin-type communication/status check, and report out the respective data. Existing systems are therefore more like an antenna on a wall with wire-based communication system to the scales/shelves, wherein only one device can talk at a time. These systems tended to be susceptible to interference from, e.g., MRI (magnetic resonance imaging) machines. Embodiments under the present disclosure, in contrast, can comprise multi-master systems which can provide IoT (internet of things) capabilities, with intelligence built-in, with ability to analyze measurements from shelves/scales, and analyze for changes that have occurred. Embodiments under the present disclosure can utilize CAN bus technology, and can avoid the interference challenges of prior art systems. Embodiments can also optimize factors such as labor costs, supply costs, or supply delivery speed. Embodiments can also predict optimized supply techniques based on AI/ML models trained on operational data.

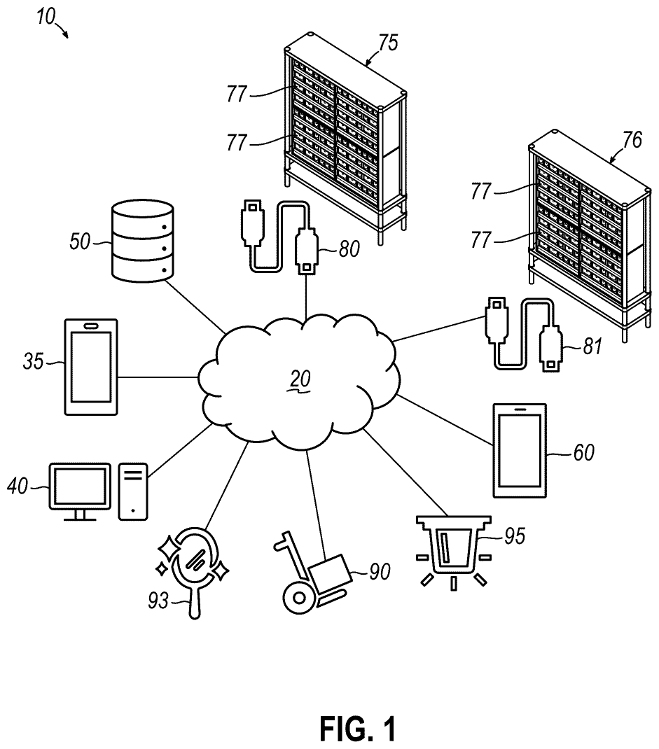

FIG. 1 illustrates one system embodiment 10 under the present disclosure. System 10 can comprise a supply/inventory tracking system at e.g., a hospital, military base, school, business, factory, or other location. Storeroom(s), cabinets, or shelves 75, 76 can store items 77 such as medicines, testing machines, needles, disinfectants, and other supplies that may be needed at e.g., a hospital. Items 77 may rest on weight sensors such as louvers or baskets that detect a weight. Each weight sensor can be coupled to scale controller 60, wirelessly or via hardwire connections 80, 81. Further details and functionalities of scale controller 60 will be discussed further below. Scale controller 60, after communicating/receiving inventory data from storerooms 75, 76, may transmit inventory data to e.g., server(s) 50. Servers 50 may be local or cloud based. Users may access inventory data via computing devices 35, 40, such as smartphones, tablets, laptops, etc. Computing devices 35, 40 may receive inventory data from servers 50, scale controller 60, or combination thereof. Network 20 may provide communicative coupling amongst various components of system 10. Network 20 can comprise the Internet, enterprise/LAN systems, cellular networks, other types of data networks, and/or combinations of any of the foregoing.

The location of the storerooms 75, 76, e.g. a hospital may comprise a plurality of sensors 95, scanners 93, or mobile carts 90, which may be able to detect the location of items 77 during transport (after arrival from a shipping company, or during transit from one storeroom to another), when opened or used (e.g., in a surgery room with a patient), or at other times. Mobile carts 90 may comprise sensors 95 and/or scanners 93 in some embodiments, and vice versa. Sensors 95 could also be located along hallways, at a delivery location, or other locations. Mobile carts 90, scanners 93, and/or sensors 95 may be wirelessly connected to network 20 and may transmit information to e.g., servers 50. Servers 50 can track, monitor, and analyze inventory data received from e.g., scale controller 60, mobile carts 90, sensors 95, scanners 93. A location (e.g., a hospital) can comprise a variety of storerooms 75, 76, located in various locations. Each storeroom 75, 76 may comprise or have its own scale controller 60, or a scale controller can be used to track multiple storerooms 75, 76.

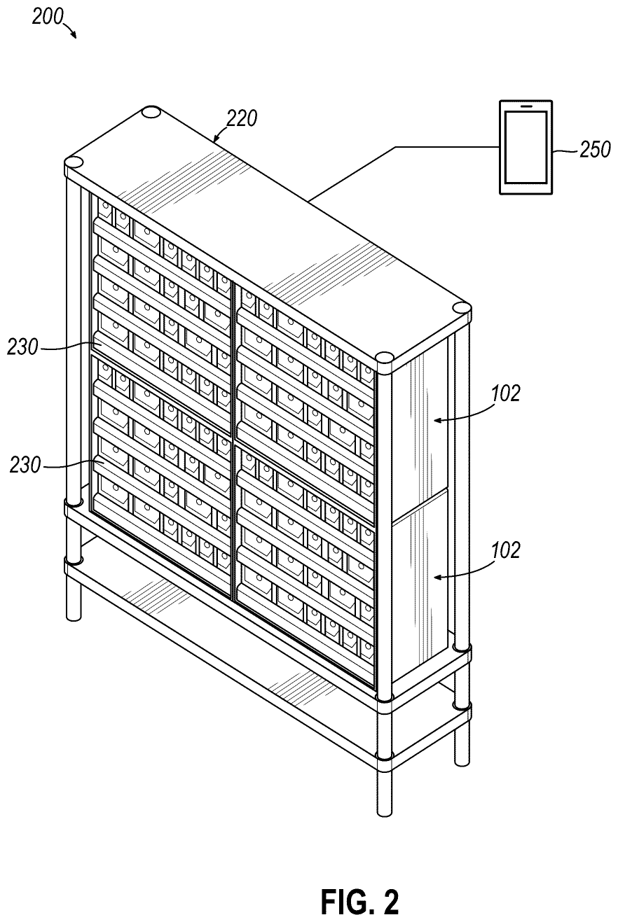

FIG. 2 illustrates a possible system 200 under the present disclosure. Here, cabinet (closet, shelf, etc.) 220 comprise a plurality of holders 230 (e.g., trays, boxes, etc.) which can retain supplies, such as hospital supplies such as bandages, thermometers, etc. Each holder 230 can be coupled within cabinet 220 with a weight sensor or scale, which can be coupled to scale controller 250. Scale controller 250 (such as scale controller 60 of FIG. 1) is preferably communicatively coupled to each holder 230 or sensor/scale that is desired to be tracked/monitored. Other embodiments can comprise wireless coupling. Each holder 230 is preferably used for one type of supply, which can be set e.g., in scale controller 250 or servers 50 of FIG. 1. If the scale controller 250 knows what type of item should be in each respective holder 230, then the data from the respective sensor/scale can be used to determine a quantity of items in a given holder 230. For example, a weight reading of 2 lbs. may correlate with a syringe quantity of 550 at a given holder 230. A weight reading of 2 lbs. in another holder 230 may correlate with a bandage quantity of 3000.

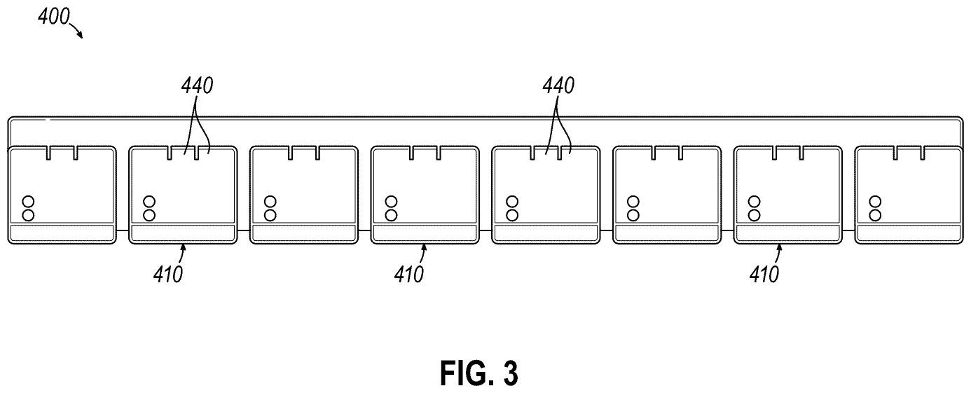

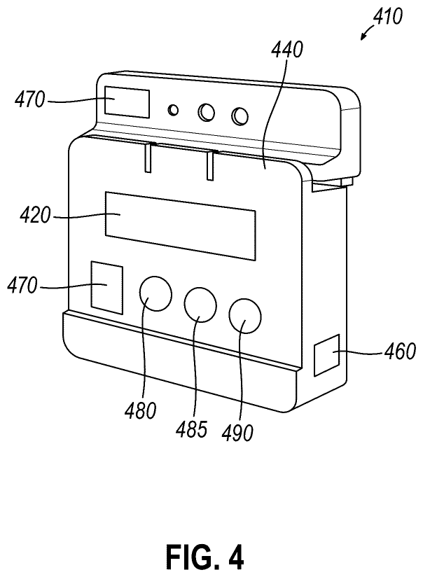

FIG. 3 illustrates a possible row 400 of sensors/scales 410. Each sensor 410 can comprise an extension 440 configured to receive a slot on a tray or holder (e.g., holder of 230 of FIG. 2). A deformation of extension 440 (or of another component within sensor 410) may indicate a weight of the respective holder when coupled to sensor 410. For example, in some embodiments sensor 410 can comprise a preloaded aluminum bar functioning as a strain gauge or load cell 420. In some embodiments sensor 410 can comprise a “conditioned” load cell. Other embodiments and types of weight sensors are possible in various embodiments. FIG. 4 illustrates a closer view of sensor or scale 410 with extension 440. Port 460 can comprise e.g., an ethernet port, or other hardwire communicative coupling means for providing communication between sensor 410 and e.g., scale controller 250 of FIG. 2. Sensor 410 can comprise one or more screens, lights, or other types of indicators 470. Some indicators 470 can comprise a light that may blink to indicate a holder status. Or in some cases some indicators 470 can comprise a screen (e.g., e-ink, touchscreen, etc.) that may indicate an item name, status, or other information. Sensor 410 can comprise an intelligent network device. It can comprise in some embodiments, analog to digital converter 480 (e.g., 24-bit), for help in converting analog signals from e.g., the load cell. Some embodiments can comprise a microcontroller 485 (e.g., 32-bit ARM microcontroller with embedded memory) for performing functions stored in memory. Some embodiments can comprise CAN transceiver 490 for communicating with e.g., scale controllers. In one sample scenario, microcontroller 485 can be programmed with a bootloader and an application. Each can update the other. The sensor 410 can independently take measurements from the load cell 420 through the ADC 480, determine if there has been a change and report that change to the scale controller which then reports to e.g., servers 50 of FIG. 1.

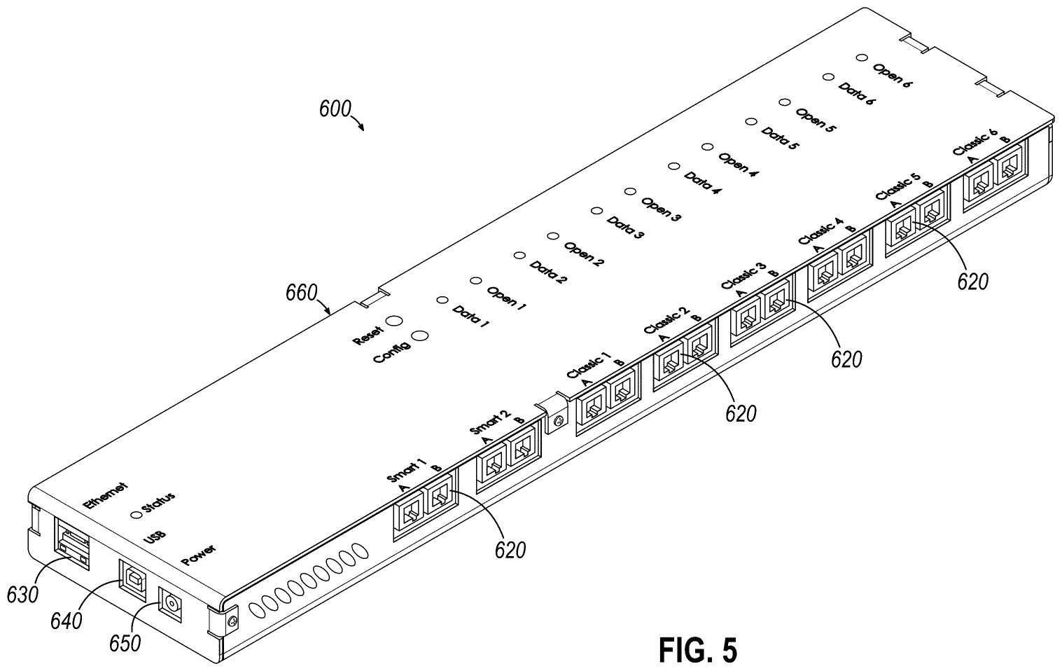

FIG. 5 illustrates a possible scale controller 600 under the present disclosure. Scale controller 600 can comprise a variety of ports 620 for hardwire coupling to e.g., sensors 410 of FIGS. 4 and 5. Scale controller 600 can also comprise ethernet port 630, USB port 640, power plug port 650, and/or other ports. Scale controller 600 can perform a variety of functions in a variety of embodiments. One function can be as a device server, managing communication between e.g., servers 50, computing devices 35, 40 of FIG. 1 and sensor/scale 410 of FIG. 4 which transmit data related to weight or supply in holders 230 of FIG. 2. In one possible bootup scenario, upon powering on, scale controller 600 can establish a connection to the servers 50 or computing devices 35, 40 of FIG. 1.

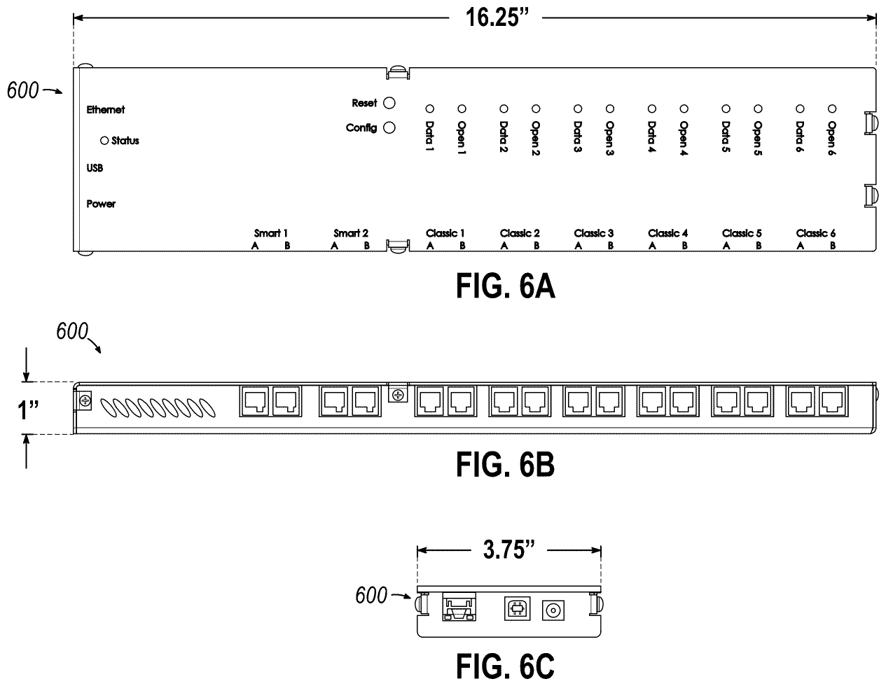

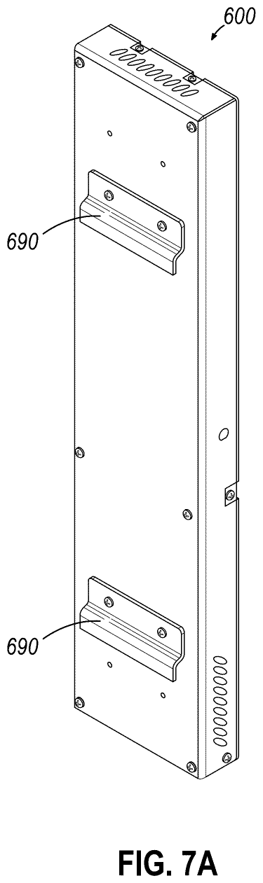

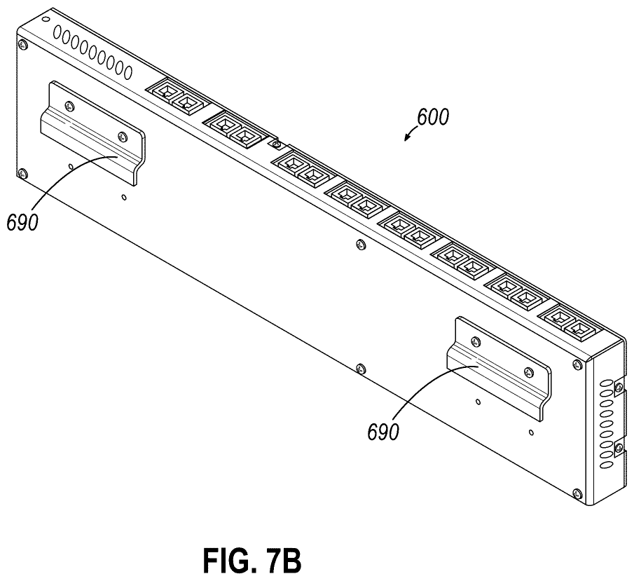

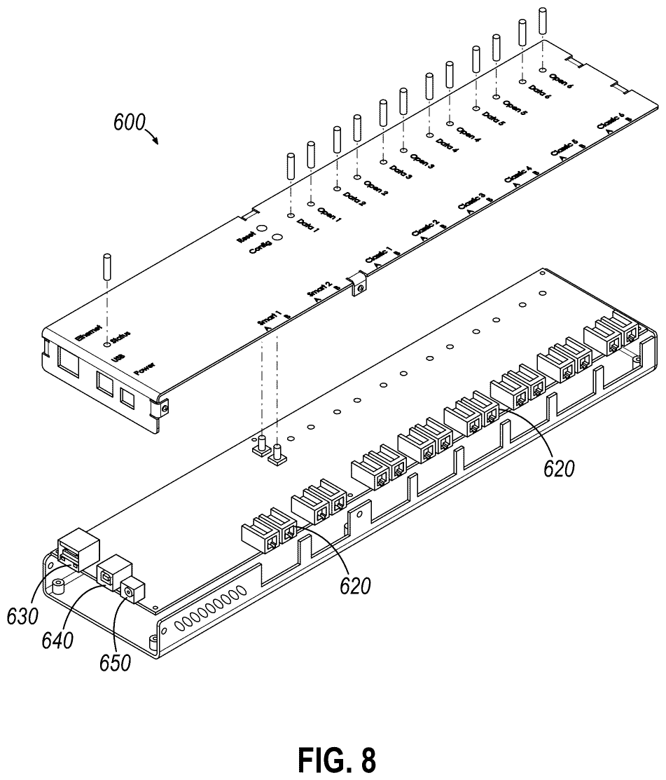

Additional views of a possible scale controller embodiment 600 are shown in FIGS. 6A to 8. Scale controller 600 can take a variety of embodiments. For example, some embodiments can comprise mounts 690, which can hook onto a latch, hook, or other means on a wall, cabinet, etc. Other mounting embodiments are possible. The following non-limiting specification embodiments can comprise some preferred variations of the present disclosure.
Size: 3¾″ (5¼″ with antenna)×16¼″×1″.
Mounting: Four #6 screws.
Power: Medical Switch Mode Power Supply—Power Partners—Model PEAD100 or equivalent.
Input: 100-240 VAC Universal Input.
Output: 12 VDC at 7.5A-2.5 mm coaxial jack
Approvals and Safety Standards: FCC Rule Part 15, Class B; CAN ICES-3 (B)/NMB-3(B).
Inputs:
   i. USB 2.1 compatible-USB Type B input
   ii. Ethernet-IEEE 802.3
   iii. 100 mbps Full Duplex
   iv. DHCP
Output
i. 6 1-wire network segments
ii. (12) RJ-12 Jacks*2 per segment-SEGMENT IN & OUT
iii. 2 Smart Network Segments
iv. (4) RJ-12 Offset Key Jacks*2 per segment-SEGMENT IN & OUT
Capacity
   i. Estimated 600 1-wire bins max (100 per segment max)
   ii. Estimated 300 Smart bins max (150 per segment max)
Indicators
   i. POWER-Power indicator
   ii. ACTIVE-Network data present
   iii. LINK—Valid ethernet link
   iv. DATA—Indicate network data traffic (1-wire)
   v. OPEN—Network open fault (1-wire).

Scale controller 600 can be preset, configured, and/or updated with an identity of items held in each tray/holder of a storeroom, supply cabinet, etc. Scale controller 600 can also know which specific scale is associated with each item, how many items can fit in a given holder, and other weight and item quantity data. The scale controller 600 can be updated or configured with this information by e.g., servers 50, computing devices 35, 40, and/or by other components capable of configuring scale controller 600. Scale controller 600, via communication with scales can be aware, even in real-time or at set periods, of how many of each item are in each holder, and therefore a status of each storeroom/closet. A location (e.g., a hospital) may comprise multiple storerooms/closets and may comprise a plurality of scale controllers 600. Servers 50 and/or computing devices 35, 40 may thereby be able to assess an inventory status for the entire location via communication with multiple scale controllers 600. When a given item is running low (e.g., below a set threshold), servers 50 or computing devices 35, 40 can be notified, or can give a notification to a user. In some embodiments servers 50 and/or computing devices 35, 40 may automatically order more of a given item.

A CAN protocol can be implemented for the communication amongst sensors/scales 410 of FIG. 4 and scale controller 600 of FIG. 5. In one example, a CAN protocol has been implemented that supports up to 511 devices on a network (one address space from the 512 is reserved for broadcasts). Devices (e.g., sensors 410) can have a built-in device-ID (e.g., called BINAADDR) that identifies the scale uniquely—this is preferably used to identify the device universally but not related to the ID of the device on the CAN network. That ID can have other titles, e.g., ScaleNet ID. The ScaleNet ID of the master device (e.g., scale controller 600) can be 0 in some embodiments. Devices that are on the network that are not the master can be called common nodes (e.g., typically sensors/scales 410). Common nodes can be assigned a ScaleNet ID which identifies their mailboxes for transmitting to the master, and receipt of single cast messages from the master or from another device. The ScaleNet ID can be arbitrated for each device by the master, but a device that does not already have a ScaleNet ID assigned (which will be the case the first time it is placed on a ScaleNet) can guess at its address by hashing its BINAADDR to the ScaleNet ID space. This might result in collisions which the master will have to resolve. ScaleNet IDs can be mapped to part of the CAN ID field, thus ensuring that only one device can actually be active on the network at a time. The lower the ID number, the higher the priority that device will incidentally have due to the way the CAN ID field is used for bus arbitration. ScaleNet ID values are saved by a scale device in Non-Volatile Memory (NVM) and used by default when a device first boots. This means that a master does not need to assign a ScaleNet address every time a device or the whole network is bounced. If a scale is moved from one ScaleNet (CAN network) to another (e.g., to another storeroom in a hospital, or to another hospital), its previously assigned ScaleNet ID, which it will have retained in its NVM, may collide with a device already installed on the device's new ScaleNet. In this case, the collision can be resolved by the Master. Upon boot, a device preferably announces its presence on the network using a Hello message. This message can have the ScaleNet ID in the CAN ID portion of the message it directs to the Master along with its BINAADDR value in the message body. If the master detects a collision of ScaleNet ID values (that is, if it finds that it already has this ScaleNet ID mapped to a different BINAADDR), then it will re-assign the ScaleNet ID for the device's given BINAADDR using a special message for this purpose. The ScaleNet mailbox design can allow for the possibility that the user might want a common node to talk directly to another common node. This could be done through a defined mailbox (e.g., mailbox 3), which can be defined but unused.

The CAN protocol can allow for a master node. In some embodiments, master node can comprise the scale controller 600, or a chosen common node (e.g., a sensor 410). The master node can act as the router/bridge to translate messages from the CAN network to the internet. But the master preferably is capable of significant processing on messages and can be responsible for things like publish-subscribe message subscription (e.g., using MQTT protocol) which the individual scales know nothing about. The master can be given control over e.g., the first 3 mailbox slots, numbering 0-2. Two can have a function that is already defined. The third can be simply reserved. These could be mapped as follows: Mailbox 0—Control transmit message from Master—Broadcast message intended for any/all recipients or directed message from the master to a common node; Mailbox 1—Directed receipt from any device specifically to the Master; Mailbox 2—Reserved and undefined.

Various possible messages and message formats are presented below as possible, but non-limiting examples, of CAN protocols under the present disclosure.

Master Transmit (Mailbox 0): [0][0][r8][r7][r6][r5][r4][r3][r2][r1][r0]. This can be used by the master to transmit a message to a recipient. R8-0 is intended recipient—0 is all recipients on the bus. Starting with 00, this ensures that messages transmitted from the master have the highest priority, no matter who the recipient is, as the CAN ID is used for arbitration by the hardware.

Master Receive (Mailbox 1): [0][1][8][7][6][5][4][s3][2][s1][s0]. This can be used by common nodes to send a message to the Master. s10-0 is sender's ScaleNet ID—this ensures that only one common node on the network can actually communicate at a time, as the CAN ID is used for arbitration by the hardware. Messages from a common node to the master have priority over any other messages, except for messages from the master to a specific node or as broadcasts. That is, the master's transmit (above) takes priority over this, and this takes priority over the other message types listed below. This is based on the way CAN ID values are used for arbitration by the hardware.

Reserved to Master—Undefined function (Mailbox 2): [1][0][bit 8-0 undefined].

Common node to common node (currently unused) (Mailbox 3): [1][1][s8][s7][s6][s5][s4][s3][s2][s1][s0]. This can be reserved to common nodes to transmit to other common nodes—Recipient is part of the payload of the remainder of the CAN frame.

Message formats (payloads) and paging descriptions examples are also provided below. Payloads can be mapped onto the data portion of the CAN frame following the arbitration (CAN ID) field. A CAN frame can have up to 8 bytes of data, with a field called Data Length Code (DLC) specifying how many there are. Example “pages” can include the following, where each byte of the 8 represents a “Line” on the page.

Measure Page. This can be sent from a scale to the master. It is the result of detection of a significant ADC change, as defined by the scale's configured threshold. It contains the ADC value, which is a 24-bit signed value. The Master interprets this command and transmits the appropriate publish-subscribe (e.g., MQTT) message to the sensor. Example: Line 1: ‘M’ (0x4D); Line 2: ADC Byte 2 (MSB); Line 3: ADC Byte 1; Line 4: ADC Byte 0 (LSB).

Measure Reply to Request Page. This can be sent from a scale to the master. It is the result of receiving a request measure message. The request identifier that was sent with the request is returned to the router so that it can pair this measure with the request. Example: Line 1: ‘m’ (0x6D); Line 2: Request identifier; Line 3: ADC Byte 2 (MSB); Line 4: ADC Byte 1; Line 5: ADC Byte 0 (LSB).

ID Page. This can be sent from a scale to the master. It is the result of detection of a double ADC spike, such as caused by double pressing on a scale, which we are using as a way to signal the identification of a scale. The master interprets this command and transmits the appropriate message to the sensor. Examples: Line 1: ‘I’ (0x49).

Threshold Page. This can be sent from a master to a scale. It is the result of the master receiving a message from the sensor indicating that a given scale should use the specified Threshold value. The message received by the master can have the BINAADDR (Device ID) of the scale. The master will translate this to the ScaleNet ID that that device has on this master's ScaleNet. The threshold given in ADC counts as the difference between measures that would be considered “significant” for that scale (based on the scale's capabilities and the weight of the items it is registered to resolve). This could be in the order of hundreds or thousands, but 24 bits have been reserved. Example: Line 1: ‘T’ (0x54); Line 2: ADC Byte 2 (MSB); Line 3: ADC Byte 1; Line 4: ADC Byte 0 (LSB).

Request Measure Page. This can be sent from a master to a scale. It is the result of the master receiving a message from the sensor indicating that a Measure is required from a given scale. This might only be used at calibration time. The message received by the master can have the BINAADDR (Device ID) of the scale. The Master will translate this to the ScaleNet ID that that device has on this master's ScaleNet. The request identifier is just a number, which will be incremented every time a request is sent, that is used by the scale in returning a measure because of a request. It allows the router to know that a particular measure message is in response to a specific request. Example: Line 1: ‘R’ (0x52); Line 2: ‘M’ (0x4D); Line 3: Request identifier.

Hello Page. This can be sent from a common node (sensor) to the master whenever it boots. It is the scale's way of letting the Master know that it exists, and how it has mapped its BINAADDR value to a ScaleNet ID value. If this device's ScaleNet is in fact unique on the network, then everything is good, and the master will simply have confirmation that this device exists. For its part, the master will ensure that it is subscribed to any messages on this scale's behalf (through the BINAADDR value). If the master sees that the ScaleNet ID value used by this BINAADDR is already mapped to a different BINAADDR, then the master will send this device a ScaleNet ID Re-Assign message to give it a newly mapped value. This message can be also broadcast to all scales from the master when it boots. When this message is received by a scale, it will reply with its own Hello message. This is the mechanism by which the master can rebuild its internal route table upon reboot. It is also possible for the master to send this message to a specific scale by specifying its ScaleNet Id value as the destination address (CAN ID). This is used to identify a scale's address if it happens to be unknown to a master any time the master receives a message from a scale. By sending it to a single scale, the master can avoid the flood of responses it would get if it broadcast the message to all scales. Example: Line 1: ‘H’ (0x48); Line 2: BINAADR byte 6 (MSB); Line 3: BINAADR byte 5; Line 4: BINAADR byte 4; Line 5: BINAADR byte 3; Line 6: BINAADR byte 2; Line 7: BINAADR byte 1; Line 8: BINAADR byte 0 (LSB).

Bootloader Hello Page. This can be sent from a common node (scale) to the master whenever it boots to the bootloader. It is the scale's way of letting the master know that it exists, its ScaleNet ID value (which can be derived in one of two ways for a bootloader), and that it is ready for a code upload. It reports the reason it decided to stay in the bootloader. In this particular case, it is possible that the ScaleNet ID is not unique on the network, and there is no way for this to be resolved. Through other considerations, it is designed that if there is a collision of ScaleNet IDs in this situation, then there will be very few devices with the same ID, to the point where it won't matter. For the bootloader, there are several ways that the ScaleNet ID is determined:
In the normal course of operation, scales are running their application. In this case, they will already have resolved their ID to a unique value. If the master wants to do a code upload, it informs the scale of this using the Firmware Update Setup Page command. When this command is received in the application, the application will mark a special place in flash with a “stay in bootloader” marker and jump to the bootloader. It will also store, in an adjacent location, the ScaleNet ID it had when running in the application. In this way the bootloader will have a unique ScaleNet ID to work with.
If the scale has never successfully been sent to a bootloader from the application, then there will be no ScaleNetID stored here. In this case, it will use its serial number to arrive at a ScaleNet ID value. This value may not be unique; however, it is unlikely to be shared by many scales on the network. This is considered a minimal risk.

A scale may be in its bootloader wanting a code upload for several reasons: it may not have a valid application, it may have been reset with its “Bootloader Button” pressed (user indicates that it should stay in bootloader), or as the result of a Firmware Update command being sent to it from the master. Example: Line 1: [symbol] ‘h’ (0x68); Line 2: <reason> (where, 0 is unknown, 1 is invalid app, 2 is bootloader button, 3 is firmware update request).

Scale Firmware Version Page. This can be sent from a common node (scale) to the master as part of the boot sequence. It identifies the version of the firmware that the scale is running. This information, at least the major and minor values, may be reported to the node/scale by the master. The test code number is intended to be used to test that a particular version of software can update itself, by allowing us to change nothing other than this number (like from a 0 to a 1) and then uploading the same code (with this change only) to a version of code. Example: Line 1: [symbol] V′ (0x56); Line 2: Major; Line 3: Minor; Line 4: Test code.

ScaleNet ID Re-Assign Page. This can be sent from a master to a scale when the master wants to Re-Assign a new ScaleNet ID to a given BINAADDR. Since a scale initially uses the ScaleNet ID is has in NVM or it guesses at the ScaleNet ID it should use if it does not already have one in NVM, this message will only be used if the master has detected a collision of ScaleNet ID values on its ScaleNet network. This is the message that the master uses to resolve such conflicts. The scale whose BINAADDR is given in the body of this message should use the ScaleNet ID given in the CAN ID portion of the message. The master will assume the device has made this assignment, but if needed, the device could reply with a new Hello message to verify it. Example: Line 1: ‘A’ (0x41); Line 2: BINAADR byte 6 (MSB); Line 3: BINAADR byte 5; Line 4: BINAADR byte 4; Line 5: BINAADR byte 3; Line 6: BINAADR byte 2; Line 7: BINAADR byte 1; Line 8: BINAADR byte 0 (LSB).

Scale Throttling. When a scale's load cell is on, it consumes a relative lot of power. Initially smart scales were designed to run all the time, constantly taking measurements, and reporting only when they detected a sufficient change. This method can be used for in some embodiments, but due to power limitations cannot be used for large networks of scales. To support large networks of scales, “throttling” can be used. Under throttling a subset of the scales on a network are allowed to take a measurement (turn on their load cells) at any given time. To manage which scales are on, Throttling Commands can be implemented as described below. Upon startup, scales preferably default to not taking measures until they hear one or the other commands from the scale controller. So, a small network used for charging will not start taking measurements until after the scale controller tells it to “Run free,” which will not occur immediately because the scale controller preferably listens for the number of scales before making that determination.

Run Free. Upon receipt, scales will simply run continuously, as originally designed, taking samples as fast as possible and reporting on changes as they detect them. This mode can preferably be used whenever there are 100 or fewer scales on a bus. To achieve the performance needed this is necessary for certain charging modes. Since a charge mode may have similar restrictions on the number of scales for 1-wire networks, this concept of limiting the number of scales is familiar practice. This limit is larger than the limit on 1-wire scales, so there is still a benefit. Example: Line 1: ‘F’ (0x46).

Throttle<nibble1>. Scales that have an address whose least significant nibble matches the provided value will take a measure and then wait for the next matching throttle command. The scale controller can cycle through these values (00-0F), thus dividing an evenly distributed set of addresses into 16 equal sections of total/16 scales. The scale controller can send the throttle command at a rate that still gives reasonably good response time for the underlying averaging algorithm in the scale. The least significant nibble in a scale address can refer to the second least significant byte. This is because scales can be serialized with a 14-character value where the least significant byte is always C0. Example: Line 1: ‘O’ (0x4F); Line 2: <nibble> (values 00-0F).

LED Control. LED control commands can be used to instruct a scale to make a pattern with its LED. This approach could be adapted for other types of displays as well. This is envisioned to be used for signaling to a user, for things such as item location or a pick count for a case. Examples uses include setting how long the LED was on and off (flash/blink) and repeat patterns. Example: Line 1: ‘L’ (0x4F); Line 2: <on for N*10 ms>; Line 3: <off for N*10 ms>; Line 4: <repeat 1>; Line 5: <pause for N*100 ms>; Line 6: <repeat 2>. For line 2, e.g., the number of 10 ms periods of ON time for the LED, 10 ms is a good granularity for what people can detect and this allows for a reasonable range of 10 ms to 2.55 seconds, 0 can be used here to turn off the LED. For line 3, the number of 10 ms periods of OFF time for the LED, 10 ms is a good granularity for what people can detect and this allows for a reasonable range of 10 ms to 2.55 seconds. This might only be important if the <repeat> count value is used. For line 4, the number of times to repeat the pattern described by 2-3 before stopping, this could be used to create a fixed pattern for a one off-like blink 5 times and then stop. Or it could be used to provide an overall timeout. For line 5, after the above pattern has been completed, it waits for this number of 100 ms periods before repeating the pattern described by line 2-4. For line 6, the number of times to repeat the pattern described by 2-5 before stopping, this could be used to create a fixed pattern for a one off-do this 4 times: blink 3 times and wait for a second in between—and then stop. Or it could be used to provide an overall timeout.

Scale Serialization. After initial programming, a scale may not have a BINAADDR value. This is because the chip may provide e.g., a 128-bit serial number which is too big to use for certain purposes. It may be unclear how to map all the bits in detail, because in some situations uniqueness is only guaranteed if all 128 bits are used. So, during implementation it may be necessary to serialize the scales again. As all communication with a scale normally happens over the CAN bus, a protocol can be created for doing this by adding commands to the ScaleNet protocol. In this case, the master is envisioned to be a special program communicating with a single scale on a CAN bus, although the possibility that multiple scales might be present on the CAN bus for this process has been taken into account. If the scale does not have a serial number, the normal method of hashing to assign itself a ScaleNet ID will not work, but to help allow for multiple devices to request a BINAADDR value on a given CAN bus in manufacturing, the 128-bit serial number can be hashed and will be used for the ScaleNet ID. There is preferably no reassignment however, since this is only used to try to eliminate the possibility of message collision on the CAN bus and is not meant to be guaranteed unique. The assignment of the BINAADDR value is based on the uniqueness of the 128-bit serial number, which will be part of the communication. Here is an example of a 128-bit serial number from a scale: Sn0: 0x97D58944; Sn1: 0x51503853; Sn2: 0x4C4A2020; Sn3: 0xFF072313.

Request BINAADDR. This can be sent from a scale to a special master. It is the result of the scale recognizing that it does not yet have a BINAADDR value (a scale serial number of the form “<12 characters>C0”). Example: Line 1: ‘r’ (0x72); Line 2: Number of pages in request (upper nibble) (will be 3), page number (lower nibble); Line 3: Serial number byte 15 (MSB)/9/3; Line 4: Serial number byte 14/8/2; Line 5: Serial number byte 13/7/1; Line 6: Serial number byte 12/6/0 (LSB); Line 7: Serial number byte 11/5; Line 8: Serial number byte 10/4.

Assign BINAADDR. This can be sent from the special master to the scale. It is the assignment of the BINAADDR by the special master. To be used, the scale will preferably match its 128-bit serial number to the value in the result and only then should it use the BINAADDR given. The scale can be listening for this based on its temporary ScaleNet ID value, which it hashed from the 128-bit serial number. After receiving this, the scale can program its new serial number and then restart its communication, which is to say it will hash its newly assigned address to derive a ScaleNet ID value and send out a Hello message. This command can be four pages long. The BINAADDR values have been interleaved with a mixed up ordering of the serial number bits that are most likely to change to hedge against the already unlikely event that a collision of the temporary ScaleNet ID value occurs if there are more than one scale being serialized on the same CAN bus. Example:
Page 1—Line 1: ‘a’ (0x61); Line 2: 0x41 [Number of pages in command (upper nibble), page number (lower nibble)]; Line 3: Serial number byte 0 (LSB); Line 4: Serial number byte 1; Line 5: Serial number byte 13; Line 6: BINAADDR byte 6 (MSB); Line 7: BINAADDR byte 5; Line 8: BINAADDR byte 4;
Page 2—Line 1: ‘a’ (0x61); Line 2: 0x42 [Number of pages in command (upper nibble), page number (lower nibble)]; Line 3: Serial number byte 2; Line 4: Serial number byte 3; Line 5: Serial number byte 12; Line 6: BINAADDR byte 3; Line 7: BINAADDR byte 2; Line 8: BINAADDR byte 1;
Page 3—Line 1: ‘a’ (0x61); Line 2: 0x43 [Number of pages in command (upper nibble), page number (lower nibble)]; Line 3: Serial number byte 15 (MSB); Line 4: Serial number byte 14; Line 5: Serial number byte 11; Line 6: Serial number byte 10; Line 7: Serial number byte 9; Line 8: BINAADDR byte 0 (LSB);
Page 4—Line 1: ‘a’ (0x61); Line 2: 0x44 [symbol] Number of pages in command (upper nibble), page number (lower nibble)]; Line 3: Serial number byte 8; Line 4: Serial number byte 7; Line 5: Serial number byte 6; Line 6: Serial number byte 5; Line 7: Serial number byte 4.

Settling Algorithm. Load cells are susceptible to external noise sources. These come in both electrical noise from electrical sources and physical vibration. When an item is added or removed, the load cell oscillates for a time afterward. Rather than just waiting a set amount of time, a settling algorithm can be used. This improves the performance and accuracy of the scale.

FreeRun/Throttling. FreeRun mode allows scales to take measurements in near real time. This is beneficial to customers who are charging for inventory items. When entering a room, the staff can enter the patient information. From that time on, items pulled will be charged to that patient. All scales are turned on and running at that point. Throttling occurs when the number of scales on a segment exceeds 100 (or another set value depending on the embodiment). This limits the bandwidth required to communicate between the scale controller and e.g., remote servers/computing devices. Each segment on a controller can operate independently in some embodiments, so it is possible to have one segment running in FreeRun and one in Throttled mode on the same controller. The scales can be set to, by default, power up in the Throttled state. If the controller determines if the CAN and 1-wire networks meet the requirements (e.g., no more than 100 scales and no more than 20 1-wire scales per segment). If the number is below the threshold, the scales can be put into a state where their load cells are always on. This increases the power use but improves the sample time. The throttling algorithm can use the scales' embedded serial number. To not overwhelm the network with traffic, one can limit the number of scales that can be communicated with at any given time using this algorithm. Scales that have an address whose least significant nibble* match the provided value will take a measure and then wait for the next matching throttle command. The scale controller can cycle through these values (00-0F), thus dividing an evenly distributed set of addresses into 16 equal sections of total/16 scales. The scale controller can send the throttle command at a rate that still gives good response time for the underlying averaging algorithm in the scale. The least significant nibble in a scale address can be the second least significant byte (because scales can in some embodiments be serialized with a 14-character value where the least significant byte is always C0). Upon startup, scales can default to not taking measures until they hear one or the other commands from the scale controller.

Scale Threshold Algorithm. The scales can use a threshold algorithm to determine if a change has occurred since the last reading. The purpose of this is to reduce the amount of network traffic on the bus. In some embodiments, the scale controller can be set (e.g., by servers 50 or computing devices 35, 40, of FIG. 1) to know what items are in what holders and what sensor/scale is associated with that item/holder. If a given item is large (e.g., a folding chair, defibrillator, etc.), then a large change in weight can be required for the sensor/scale or scale controller to detect a change in status. For example, if a given item weighs 100 lbs., then a 1 lb. change in weight can be set to not indicate a status or supply change.

Firmware distribution. Firmware updates can involve the transmission of blocks of data over the CAN protocol, which may necessitate using many CAN frames. In this implementation embodiment, the master always initiates this process, and it will do so in response to receiving an update from an OTA (over the air) server for the firmware in the scales. The master is responsible for polling the OTA server for scale firmware updates (as well as its own update), and for delivering the update to the scales. The overall design is predicated on the idea that every scale on a given ScaleNet (all the scales served by a master) will have the same firmware version, and that the master is able to download and store it local to itself and then deliver it to the scales (as opposed to somehow streaming it from the internet to the scales without storing it locally, although this design doesn't necessarily preclude that). When delivering scale updates, the broadcast address can be used, so that all the scales receive the same data at the same time. It is possible for an update to be delivered to a specific scale on the ScaleNet, something that may need to be done if a particular scale does not take an update properly during a broadcast. The scale controller tells the nodes who it has picked to be responsible for ACKing (acknowledging) the packets by including that scale's ScaleNet ID in the Firmware Update Setup message.

Firmware Update (Scale side). This can be sent from the master to all common nodes (usually, using the broadcast address of 0, but it could potentially be directed to a specific ScaleNet ID). This page can be used to tell the scales that a firmware update is coming, and how many bytes (lines) they will be receiving. If it becomes desirable or necessary in the future to perform firmware upgrades in a streaming fashion, filling the length bytes with all Is could be done to indicate that the length is unknown or indeterminate. In this case, the “end of stream” could be indicated by sending an empty block page as indicated below. It may be necessary for blocks to be acknowledged, to provide pacing so as not to have hundreds of nodes acknowledging each packet. The master node can decide and indicate which single node is responsible for doing this. This node can be called the “Reply partner” and is indicated by sending the ScaleNet ID for the node as part of this page. The scales can stay in the bootloader at startup (reboot or reset) so that the controller/host can force an update to a scale that may have a corrupted or incorrect application. This is an “anti-brick” feature to keep the scales from getting stuck with bad code and no way to force an update. The scale specified as the reply partner NACKs (negative acknowledges) the Firmware Update-Setup Page command. If the reply partner is set to 0 initially, then multiple scales can ACK this command. The host can use the first one and send out the command again with the reply partner value specified. The ACK that is sent back is the same as the Block Ack Page, but with the page set to 0. Example: Line 1: ‘U’ (0x55); Line 2: Line count byte 3 (MSB); Line 3: Line count byte 2; Line 4: Line count byte 1; Line 5: Line count byte 0 (LSB); Line 6: Reply partner ScaleNetID byte 1 (MSB); Line 7: Reply partner ScaleNetID byte 0 (LSB).

Block Page (0x81, 0x82, . . . , 0x8F). This page can be used as a generic block transfer section page. Blocks are sent alternating using an 0x80 base with a block number ORed onto it, ranging from 1 to 15 (0x01 to 0x0F). 7 bytes of actual payload can be delivered by each one. In some embodiments the firmware update uses these as well. Block pages can normally be fully loaded, meaning they contain 7 bytes of actual payload. The final block page could be truncated or padded with 0s depending on the application. In the case of firmware updates, the number of bytes can be known ahead of time, so the application can just use what it knows to be valid. Each CAN frame can indicate the number of bytes in the CAN Data field, which is where these pages are designed to fit, so a truncated (non-padded) block is easily accommodated. An “end of stream” for future use can send an 0x8<n> block that has no further payload. Example: Line 1: 0x81, 0x82, . . . 0x8F; Line 2: Byte 0 [optional]; Line 3: Byte 1 [optional]; Line 4: Byte 2 [optional]; Line 5: Byte 3 [optional]; Line 6: Byte 4 [optional]; Line 7: Byte 5 [optional]; Line 8: Byte 6 [optional].

Block 0x80 ACK Page (0x80). After each block is transferred, a single ACK can be expected, one to one. Example: Line 1: 0x80; Line 2: Block number (0x0<n>, where n is 1-15—so 0x01-0x0F).

Firmware Update-Completion/CRC Page. This can be sent from the master to all common nodes (usually, using the broadcast address of 0, but can be directed to a specific ScaleNet ID). This page can be used to tell the scales that a firmware update is now complete. If it was a successful upload, it provides the controller for the data that was transmitted through all the blocks. If the controller matches the scale's own calculation the application uploaded will be marked valid and the scale will reboot to it. If it was not a successful upload, from the master's perspective, then the completion page will be sent without a CRC (cyclic redundancy check). This is to be used by the scale bootloader to abandon the firmware update and exit the bootloader (which, because the application space will be corrupted, will just result in rebooting back into the bootloader, but everything will be reset and ready for another try). Example Format 1: Upload successful, CRC provided: Line 1: ‘u’ (0x75); Line 2: CRC byte 1 (MSB); Line 3: CRC byte 0 (LSB). Example Format 2: Upload unsuccessful, no CRC provided: Line 1: ‘u’ (0x75).

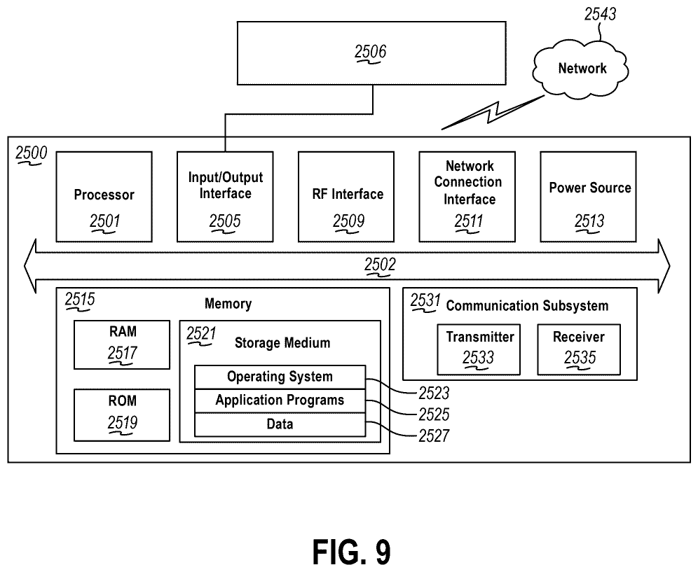

FIG. 9 illustrates a possible embodiment of various possible computing devices or any processor-based components within scale monitoring system 10 of FIG. 1, or components thereof e.g., servers 50, computing devices 35, 40, sensor 95, mobile cart 90, and/or scanner 93 of FIG. 1; scale controller 600 of FIG. 5; sensor/scale 410 of FIG. 4; or other computing or smart devices described herein. FIG. 9 shows a schematic block diagram of a computing device 2500 (or components thereof) according to certain embodiments of the present disclosure.

Computing device 2500 includes processor 2501 that is operatively coupled via a bus 2502 to an input/output interface 2505, a power source 2513, a memory 2515, a RF interface 2509, network communication interface 2511, and/or any other component, or any combination thereof. The level of integration between the components may vary from one embodiment to another. Further, certain computing devices 2500 (or components thereof) may contain multiple instances of a component, such as multiple processors, memories, transceivers, transmitters, receivers, etc.

The processor 2501 is configured to process instructions and data and may be configured to implement any sequential state machine operative to execute instructions stored as machine-readable computer programs in memory 2515. Processor 2501 may be implemented as one or more hardware-implemented state machines (e.g., in discrete logic, field-programmable gate arrays (FPGAs), application specific integrated circuits (ASICs), etc.); programmable logic together with appropriate firmware; one or more stored computer programs, general-purpose processors, such as a microprocessor or digital signal processor (DSP), together with appropriate software; or any combination of the above. For example, the processor 2501 may include multiple central processing units (CPUs).

In the example, input/output interface 2505 may be configured to provide an interface or interfaces to an input/output device(s) 2506, such as a screen, keyboard, indicator light, keypad, touchscreen, or other input or output device. Other examples of an output device include a speaker, a sound card, a video card, a display, a monitor, a printer, an actuator, an emitter, a smartcard, another output device, or any combination thereof. An input device may allow a user to capture information into system 2500. Other examples of an input device include a touch-sensitive or presence-sensitive display, a camera (e.g., a digital camera, a digital video camera, a web camera, etc.), a microphone, a sensor, a mouse, a trackball, a directional pad, a trackpad, a scroll wheel, a smartcard, and the like. The presence-sensitive display may include a capacitive or resistive touch sensor to sense input from a user. A sensor may be, for instance, an accelerometer, a gyroscope, a tilt sensor, a force sensor, a magnetometer, an optical sensor, a proximity sensor, a biometric sensor, etc., or any combination thereof. An output device may use the same type of interface port as an input device. For example, a Universal Serial Bus (USB) port may be used to provide an input device and an output device.

In some embodiments, the power source 2513 is structured as a battery or battery pack. Other types of power sources, such as an external power source (e.g., an electricity outlet), photovoltaic device, or power cell, may be used. The power source 2513 may further include power circuitry for delivering power from the power source 2513 itself, and/or an external power source, to the various parts of computing device 2500 via input circuitry or an interface such as an electrical power cable.

Memory 2515 may be configured to include memory such as random-access memory (RAM) 2517, read-only memory (ROM) 2519, programmable read-only memory (PROM), erasable programmable read-only memory (EPROM), electrically erasable programmable read-only memory (EEPROM), magnetic disks, optical disks, hard disks, removable cartridges, flash drives, other storage medium 2521, and so forth. In one example, the memory 2515 includes one or more application programs 2525, an operating system 2523, web browser application, a widget, gadget engine, or other application, and corresponding data 2527. Memory 2515 may store, for use by the computing device 2500, any of a variety of various operating systems or combinations of operating systems. An article of manufacture, such as one including a simulation system or communication system may be tangibly embodied as or in memory 2515, which may be or comprise a device-readable storage medium.

Processor 2501 may be configured to communicate with an access network or other network using the RF interface 2509 or network connection interface 2511. The RF interface 2509 or network connection interface 2511 may comprise one or more communication subsystems and may include or be communicatively coupled to an antenna. In the illustrated embodiment, communication functions of the RF interface 2509 or network connection interface 2511 may include cellular communication, Wi-Fi communication, LPWAN communication, data communication, voice communication, multimedia communication, short-range communications such as Bluetooth, near-field communication, location-based communication such as the use of the global positioning system (GPS) to determine a location, another like communication function, or any combination thereof.

Computing devices under the present disclosure, such as e.g., servers 50, computing devices 35, 40, sensor 95, mobile cart 90, and/or scanner 93 of FIG. 1; scale controller 600 of FIG. 5; sensor/scale 410 of FIG. 4, can perform ML-based methods such as described herein, e.g., enhancing, optimizing, scheduling, and implementing supply processes, such as supply delivery, choosing of workers to perform supply-related tasks, choosing of delivery/stocking routes within a building (such as a hospital), and other tasks. In certain embodiments a computing device under the present disclosure can comprise a ML/AI engine in for training or implementing a ML/AI model. The architecture of an ML model (e.g., structure, number of layers, nodes per layer, activation function etc.) may need to be tailored for each particular use case. For example, properties to vary can include e.g.: geo-location data, routes, room numbers or identifiers, ASN (autonomous system number), shipping/tracking numbers from e.g., FedEx, nurse call-down data (data related to nurses or other workers calling for more supplies), scale controller or sensor data, detour data (e.g., elevator or stair closures within a building/location), or other supply data or building/location-related data. These may all need to be considered when designing the ML model's architecture. Using the AI/ML functionality described herein, a location (e.g., a hospital) may be able to optimize various supply-related outcomes. For example, by analyzing data on supply delivery, the hospital may be able to determine which workers are most efficient at stocking supply rooms, which routes through a hospital are quickest or more efficient, what times of day are best, what days or times of day for mail deliveries are best, what impacts a detour may create (e.g. an elevator closure) or a variety of other factors and outcomes can be analyzed and optimized. This AI/ML functionality can be built around collection of data from any or all of various components of FIG. 1, e.g., servers 50, computing devices 35, 40, sensor 95, mobile cart 90, and/or scanner 93 of FIG. 1; scale controller 600 of FIG. 5; sensor/scale 410 of FIG. 4.

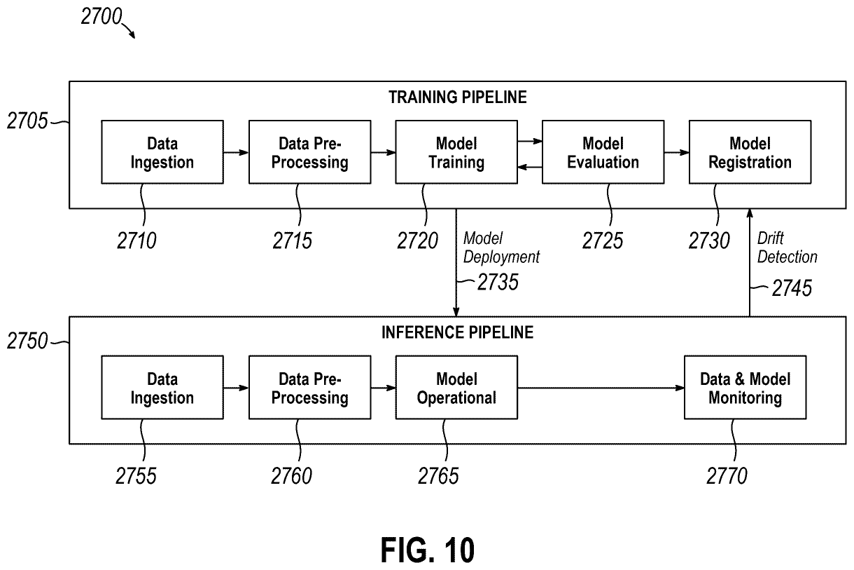

Building an AI/ML model includes several development steps where the actual training of the ML model is just one step in a training pipeline. An important part in AI/ML development is the AI/ML model lifecycle management. One embodiment of a model lifecycle management procedure 2700 is illustrated in FIG. 10. The model lifecycle management comprises two pipelines: a training pipeline 2705 and an inference pipeline 2750.

At 2710 in the training pipeline 2705, data ingestion 2710 occurs, which includes gathering raw (training) data from a data storage, e.g., servers 50 of FIG. 1, which may store any type of data related to the performance or history of system 10 of FIG. 1, or datasets for training an AI/ML model. After data ingestion 2710, there may also be a step that controls the validity of the gathered data. At 2715 data pre-processing occurs, which can include feature engineering applied to the gathered data. This may involve, e.g., data normalization or data formatting or transformation required for the input data to the AI/ML model. After the ML model's architecture is fixed, it should be trained on one or more datasets. At 2720 model training is performed in which the AI/ML model is trained with the raw training data. To achieve good performance during live operation in a system (the so-called inference phase), the training datasets should be representative of actual data the ML model will encounter during live operation. The training process often involves numerically tuning the ML model's trainable parameters (e.g., the weights and biases of the underlying neural network (NN)) to minimize a loss function on the training datasets. The loss function may be based on, for example, a maximum/minimum spend on tools or supplies, a maximum/minimum speed of performing a task, speed of restocking supply rooms, spend on employee wages, or other outputs. The purpose of the loss function is to meaningfully quantify the reconstruction error for the particular use case at hand. At 2725 model evaluation can be performed where the performance is benchmarked to some baseline. Model training 2720 and evaluation 2725 can be iterated until an acceptable level of performance is achieved. At 2730 model registration occurs, in which the AI/ML model is registered with any corresponding data on how the AI/ML model was developed, and e.g., AI/ML model evaluation data. At 2735 model deployment occurs, wherein the trained/re-trained AI/ML model is implemented in the inference pipeline 2750.

Data ingestion 2755 in the inference pipeline 2750 refers to gathering raw (inference) data from a data source. Data pre-processing 2760 can be essentially identical/similar to the data pre-processing 2715 of the training pipeline 2705. At 2765, the operational model received from the training pipeline 2705 is used to process new data received during operation of e.g., system 10 of FIG. 1. At 2770 data and model monitoring is performed. Here the inference data is analyzed to determine whether the inference data are from a distribution that aligns with the training data, as well as monitoring model outputs for detecting any performance, or operational, variance or drifts. The variance or drift is used at 2745 (drift detection) to update the AI/ML model registration.

The training process is typically based on some variant of a gradient descent algorithm, which, at its core, can comprise three components: a feedforward step, a back propagation step, and a parameter optimization step. These steps can be described using a dense ML model (i.e., a dense NN with a bottleneck layer) as an example.

Feedforward: A batch of training data, such as a mini-batch, (e.g., several downlink-channel estimates) is pushed through the ML model, from the input to the output. The loss function is used to compute the reconstruction loss for all training samples in the batch. The reconstruction loss may be an average reconstruction loss for all training samples in the batch.

Back propagation (BP): The gradients (partial derivatives of the loss function with respect to each trainable parameter in the ML model) are computed. The back propagation algorithm sequentially works backwards from the ML model output, layer-by-layer, back through the ML model to the input. The back propagation algorithm is built around the chain rule for differentiation: When computing the gradients for layer n in the ML model, it uses the gradients for layer n+1.

Parameter optimization: The gradients computed in the back propagation step are used to update the ML model's trainable parameters. It is preferred to make small adjustments to each parameter with the aim of reducing the average loss over the (mini) batch. It is common to use special optimizers to update the ML model's trainable parameters using gradient information. The following optimizers are widely used to reduce training time and improving overall performance: adaptive sub-gradient methods (AdaGrad), RMSProp, and adaptive moment estimation (ADAM).

The above process (feedforward, back propagation, parameter optimization) is repeated many times until an acceptable level of performance is achieved on the training dataset. An acceptable level of performance may refer to the ML model achieving a pre-defined average reconstruction error over the training dataset (e.g., normalized MSE of the reconstruction error over the training dataset is less than, say, 0.1). Alternatively, it may refer to the ML model achieving a pre-defined value chosen by a user.

In some implementations, a function F(⋅) may be generated by a ML process, such as, for example, supervised learning, reinforcement learning, and/or unsupervised learning. It should further be understood that supervised learning may be done in various ways, such as, for example, using random forests, support vector machines, neural networks, and the like. By way of non-limiting example, any of the following types of neural networks that may be utilized, including, deep neural networks (DNNs), convolutional neural networks (CNNs), and recurrent neural networks (RNNs), or any other known or future neural network that satisfies the needs of the system. In an implementation using supervised learning the neural networks may be easily integrated into the hardware described in inventory system 50 of FIG. 1 (e.g., in the form of simple vector-matrix multiplications).

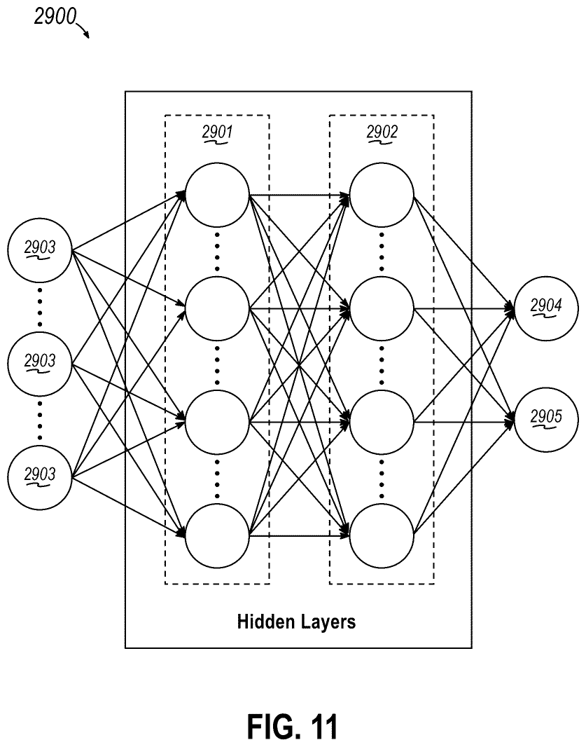

Referring now to FIG. 11, an example NN 2900 (e.g., DNN) is shown. In some implementations, and as shown, the neural network 2900 may include two hidden layers represented by dashed boxes 2901 and 2902. In one implementation, the inputs 2903 may be fed into the NN 2900. Next, the inputs 2403 may go through a set of hidden layers (e.g., 2901 and/or 2902). Once the inputs 2903 pass though the hidden layers 2901 and/or 2902, they may be output (e.g., as an output layer) as e.g., supply costs, labor costs, restocking speed, combinations of the foregoing, or other outputs desired to be optimized. Possible inputs can include e.g.: geo-location data of workers or supplies, routes, room numbers or identifiers, ASN, shipping/tracking numbers, nurse call-down data, scale controller or sensor data, detour data, or other supply data or building/location-related data.

As should be understood by one of ordinary skill in the art, in order for the NN 2900 to output proper a proper analysis, it should be trained properly (e.g., with a collection of samples) to accurately extract the likelihood values. If not trained properly, overfitting (e.g., when the NN memorizes the structure of the preambles but is unable to generalize to unseen preamble characteristics) or underfitting (e.g., when the NN is unable to learn a proper function even on the data that it was trained on) may happen. Thus, implementations may exist that prevent overfitting or underfitting, involving a set of well-engineered features that must be extracted from the preamble characteristics.

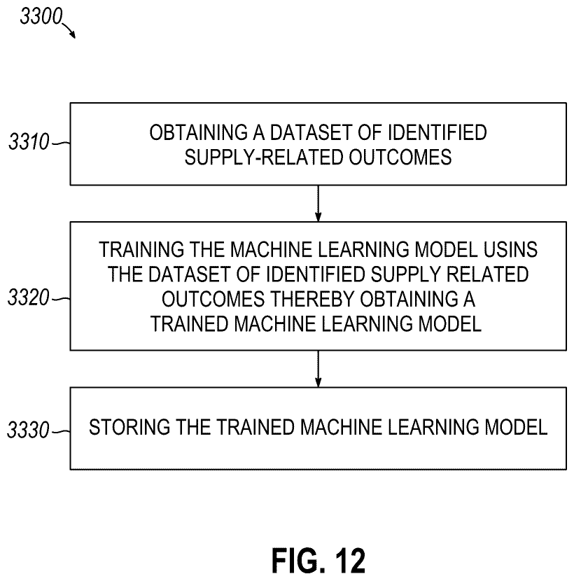

FIG. 12 displays a possible method embodiment under the present disclosure. Method 3300 is a computer implemented method for training a machine learning model for optimizing supply delivery. Step 3310 is obtaining a dataset of identified supply-related outcomes. Step 3320 is training the machine learning model using the dataset of identified supply-related outcomes thereby obtaining a trained machine learning model. Step 3330 is storing the trained machine learning model. Method 3300 can comprise a variety of additional, alternative, or optional steps or other variations. For example, further steps can comprise training a machine learning model for optimizing identified supply-related outcomes, wherein the training comprises; training the machine learning model using a dataset of one or more identified supply-related outcomes, thereby obtaining a further trained machine learning model; and storing the further trained machine learning model. Other embodiments of method 3300 can comprise any of the steps of method 3700, described below.

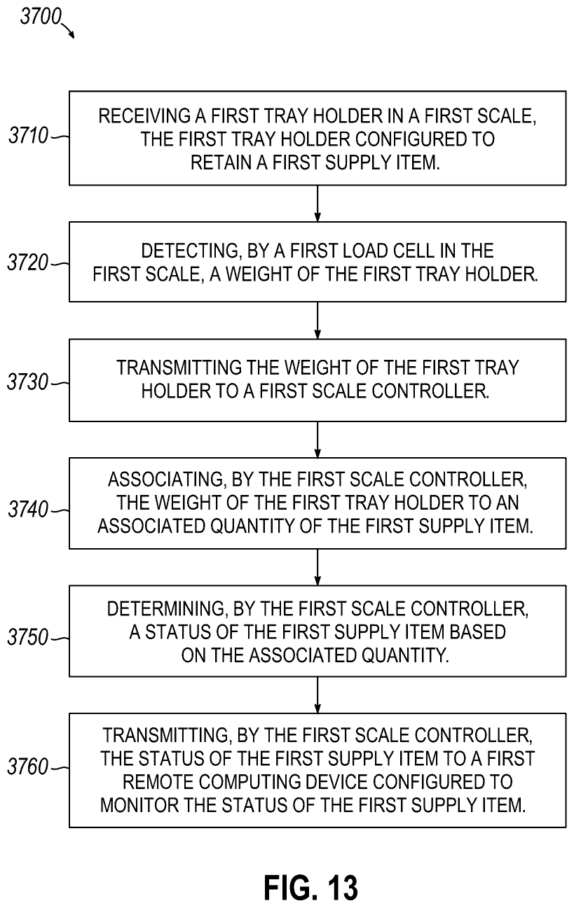

FIG. 13 illustrates a possible method embodiment under the present disclosure. Method 3700 is a method of monitoring supply items. Step 3710 is receiving a first tray holder in a first scale, the first tray holder configured to retain a first supply item. Step 3720 is detecting, by a first load cell in the first scale, a weight of the first tray holder. Step 3730 is transmitting the weight of the first tray holder to a first scale controller. Step 3740 is associating, by the first scale controller, the weight of the first tray holder to an associated quantity of the first supply item. Step 3750 is determining, by the first scale controller, a status of the first supply item based on the associated quantity. Step 3760 is transmitting, by the first scale controller, the status of the first supply item to a first remote computing device configured to monitor the status of the first supply item. Method 3700 can comprise a variety of additional, alternative and/or optional steps. For example, in some embodiments the first load cell comprises a strain gauge. Some embodiments further comprise: receiving a second tray holder in a second scale, the second tray holder configured to retain a second supply item; detecting, by a second load cell in the second scale, a weight of the second tray holder; transmitting the weight of the second tray holder to the first scale controller; associating, by the first scale controller, the weight of the second tray holder to an associated quantity of the second supply item; determining, by the first scale controller, a status of the second supply item based on the associated quantity; and transmitting, by the first scale controller, the status of the second supply item to a first remote computing device configured to monitor the status of second first supply item. In some embodiments, the first scale controller is coupled to the first scale via at least one of: a wireless connection; a hard wire connection. In some embodiments, the first scale controller is coupled to the first remote computing device via at least one of: a wireless connection; a hard wire connection. Some embodiments further comprise: transmitting, by the first remote computing device, a notification to a user when the status of the first supply item is low. Some embodiments can combine any of the steps of method 3700 (or any variations thereof) with any of the steps of method 3300 (or any variations thereof).

Although the computing devices described herein (e.g., scale controllers, scales, servers, computing devices, etc.) may include the illustrated combination of hardware components, other embodiments may comprise computing devices with different combinations of components. It is to be understood that these computing devices may comprise any suitable combination of hardware and/or software needed to perform the tasks, features, functions, and methods disclosed herein. Determining, calculating, obtaining or similar operations described herein may be performed by processing circuitry, which may process information by, for example, converting the obtained information into other information, comparing the obtained information or converted information to information stored in the network node, and/or performing one or more operations based on the obtained information or converted information, and as a result of said processing making a determination. Moreover, while components are depicted as single boxes located within a larger box, or nested within multiple boxes, in practice, computing devices may comprise multiple different physical components that make up a single illustrated component, and functionality may be partitioned between separate components. For example, a communication interface may be configured to include any of the components described herein, and/or the functionality of the components may be partitioned between the processing circuitry and the communication interface. In another example, non-computationally intensive functions of any of such components may be implemented in software or firmware and computationally intensive functions may be implemented in hardware.

In certain embodiments, some or all of the functionality described herein may be provided by processing circuitry executing instructions stored on in memory, which in certain embodiments may be a computer program product in the form of a non-transitory computer-readable storage medium. In alternative embodiments, some or all of the functionality may be provided by the processing circuitry without executing instructions stored on a separate or discrete device-readable storage medium, such as in a hard-wired manner. In any of those particular embodiments, whether executing instructions stored on a non-transitory computer-readable storage medium or not, the processing circuitry can be configured to perform the described functionality. The benefits provided by such functionality are not limited to the processing circuitry alone or to other components of the computing device but are enjoyed by the computing device as a whole, and/or by end users and a wireless network generally.

It will be appreciated that computer systems are increasingly taking on a wide variety of forms. In this description and in the claims, the terms “controller,” “computer system,” or “computing system” are defined broadly as including any device or system—or combination thereof—that includes at least one physical and tangible processor and a physical and tangible memory capable of having thereon computer-executable instructions that may be executed by a processor. By way of example, not limitation, the term “computer system” or “computing system,” as used herein is intended to include personal computers, desktop computers, laptop computers, tablets, hand-held devices (e.g., mobile telephones, PDAs, pagers), microprocessor-based or programmable consumer electronics, minicomputers, mainframe computers, multi-processor systems, network PCs, distributed computing systems, datacenters, message processors, routers, switches, and even devices that conventionally have not been considered a computing system, such as wearables (e.g., glasses).

The computing system also has thereon multiple structures often referred to as an “executable component.” For instance, the memory of a computing system can include an executable component. The term “executable component” is the name for a structure that is well understood to one of ordinary skill in the art in the field of computing as being a structure that can be software, hardware, or a combination thereof. For instance, when implemented in software, one of ordinary skill in the art would understand that the structure of an executable component may include software objects, routines, methods, and so forth, that may be executed by one or more processors on the computing system, whether such an executable component exists in the heap of a computing system, or whether the executable component exists on computer-readable storage media. The structure of the executable component exists on a computer-readable medium in such a form that it is operable, when executed by one or more processors of the computing system, to cause the computing system to perform one or more functions, such as the functions and methods described herein. Such a structure may be computer-readable directly by a processor—as is the case if the executable component were binary. Alternatively, the structure may be structured to be interpretable and/or compiled—whether in a single stage or in multiple stages—so as to generate such binary that is directly interpretable by a processor.

The terms “component,” “service,” “engine,” “module,” “control,” “generator,” or the like may also be used in this description. As used in this description and in this case, these terms—whether expressed with or without a modifying clause—are also intended to be synonymous with the term “executable component” and thus also have a structure that is well understood by those of ordinary skill in the art of computing.

In terms of computer implementation, a computer is generally understood to comprise one or more processors or one or more controllers, and the terms computer, processor, and controller may be employed interchangeably. When provided by a computer, processor, or controller, the functions may be provided by a single dedicated computer or processor or controller, by a single shared computer or processor or controller, or by a plurality of individual computers or processors or controllers, some of which may be shared or distributed. Moreover, the term “processor” or “controller” also refers to other hardware capable of performing such functions and/or executing software, such as the example hardware recited above.

In general, the various exemplary embodiments may be implemented in hardware or special purpose chips, circuits, software, logic, or any combination thereof. For example, some aspects may be implemented in hardware, while other aspects may be implemented in firmware or software which may be executed by a controller, microprocessor, or other computing device, although the disclosure is not limited thereto. While various aspects of the exemplary embodiments of this disclosure may be illustrated and described as block diagrams, flow charts, or using some other pictorial representation, it is well understood that these blocks, apparatus, systems, techniques, or methods described herein may be implemented in, as non-limiting examples, hardware, software, firmware, special purpose circuits or logic, general purpose hardware or controller or other computing devices, or some combination thereof.

While not all computing systems require a user interface, in some embodiments a computing system includes a user interface for use in communicating information from/to a user. The user interface may include output mechanisms as well as input mechanisms. The principles described herein are not limited to the precise output mechanisms or input mechanisms as such will depend on the nature of the device. However, output mechanisms might include, for instance, speakers, displays, tactile output, projections, holograms, and so forth. Examples of input mechanisms might include, for instance, microphones, touchscreens, projections, holograms, cameras, keyboards, stylus, mouse, or other pointer input, sensors of any type, and so forth.

### Abbreviations and Defined Terms

To assist in understanding the scope and content of this written description and the appended claims, a select few terms are defined directly below. Unless defined otherwise, all technical and scientific terms used herein have the same meaning as commonly understood by one of ordinary skill in the art to which the present disclosure pertains.

The terms “approximately,” “about,” and “substantially,” as used herein, represent an amount or condition close to the specific stated amount or condition that still performs a desired function or achieves a desired result. For example, the terms “approximately,” “about,” and “substantially” may refer to an amount or condition that deviates by less than 10%, or by less than 5%, or by less than 1%, or by less than 0.1%, or by less than 0.01% from a specifically stated amount or condition.

Various aspects of the present disclosure, including devices, systems, and methods may be illustrated with reference to one or more embodiments or implementations, which are exemplary in nature. As used herein, the term “exemplary” means “serving as an example, instance, or illustration,” and should not necessarily be construed as preferred or advantageous over other embodiments disclosed herein. In addition, reference to an “implementation” of the present disclosure or embodiments includes a specific reference to one or more embodiments thereof, and vice versa, and is intended to provide illustrative examples without limiting the scope of the present disclosure, which is indicated by the appended claims rather than by the present description.

As used in the specification, a word appearing in the singular encompasses its plural counterpart, and a word appearing in the plural encompasses its singular counterpart, unless implicitly or explicitly understood or stated otherwise. Thus, it will be noted that, as used in this specification and the appended claims, the singular forms “a,” “an” and “the” include plural referents unless the context clearly dictates otherwise. For example, reference to a singular referent (e.g., “a widget”) includes one, two, or more referents unless implicitly or explicitly understood or stated otherwise. Similarly, reference to a plurality of referents should be interpreted as comprising a single referent and/or a plurality of referents unless the content and/or context clearly dictate otherwise. For example, reference to referents in the plural form (e.g., “widgets”) does not necessarily require a plurality of such referents. Instead, it will be appreciated that independent of the inferred number of referents, one or more referents are contemplated herein unless stated otherwise.

References in the specification to “one embodiment,” “an embodiment,” “an example embodiment,” and the like indicate that the embodiment described may include a particular feature, structure, or characteristic, but it is not necessary that every embodiment includes the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

It shall be understood that although the terms “first” and “second” etc. may be used herein to describe various elements, these elements should not be limited by these terms. These terms are only used to distinguish one element from another. For example, a first element could be termed a second element, and similarly, a second element could be termed a first element, without departing from the scope of example embodiments. As used herein, the term “and/or” includes any and all combinations of one or more of the associated listed terms.

It will be further understood that the terms “comprises”, “comprising”, “has”, “having”, “includes” and/or “including”, when used herein, specify the presence of stated features, elements, and/or components etc., but do not preclude the presence or addition of one or more other features, elements, components and/or combinations thereof.

### CONCLUSION

The present disclosure includes any novel feature or combination of features disclosed herein either explicitly or any generalization thereof. Various modifications and adaptations to the foregoing exemplary embodiments of this disclosure may become apparent to those skilled in the relevant arts in view of the foregoing description, when read in conjunction with the accompanying drawings. However, any and all modifications will still fall within the scope of the non-limiting and exemplary embodiments of this disclosure.

It is understood that for any given component or embodiment described herein, any of the possible candidates or alternatives listed for that component may generally be used individually or in combination with one another, unless implicitly or explicitly understood or stated otherwise. Additionally, it will be understood that any list of such candidates or alternatives is merely illustrative, not limiting, unless implicitly or explicitly understood or stated otherwise.

In addition, unless otherwise indicated, numbers expressing quantities, constituents, distances, or other measurements used in the specification and claims are to be understood as being modified by the term “about,” as that term is defined herein. Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

Any headings and subheadings used herein are for organizational purposes only and are not meant to be used to limit the scope of the description or the claims. The terms and expressions which have been employed herein are used as terms of description and not of limitation, and there is no intention in the use of such terms and expressions of excluding any equivalents of the features shown and described or portions thereof, but it is recognized that various modifications are possible within the scope of the present disclosure. Thus, it should be understood that although the present disclosure has been specifically disclosed in part by certain embodiments, and optional features, modification and variation of the concepts herein disclosed may be resorted to by those skilled in the art, and such modifications and variations are considered to be within the scope of this present description.

It will also be appreciated that systems, devices, products, kits, methods, and/or processes, according to certain embodiments of the present disclosure may include, incorporate, or otherwise comprise properties or features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

It will be apparent to one of ordinary skill in the art that methods, devices, device elements, materials, procedures, and techniques other than those specifically described herein can be applied to the practice of the described embodiments as broadly disclosed herein without resort to undue experimentation. All art-known functional equivalents of methods, devices, device elements, materials, procedures, and techniques specifically described herein are intended to be encompassed by this present disclosure.

When a group of materials, compositions, components, or compounds is disclosed herein, it is understood that all individual members of those groups and all subgroups thereof are disclosed separately. When a Markush group or other grouping is used herein, all individual members of the group and all combinations and sub-combinations possible of the group are intended to be individually included in the disclosure.

The above-described embodiments are examples only. Alterations, modifications, and variations may be affected to the particular embodiments by those of skill in the art without departing from the scope of the description, which is defined solely by the appended claims.

What is claimed is:

1. A system for monitoring a supply inventory, the system comprising:
a first scale comprising a first load cell and configured to receive a first tray holder, the first tray holder configured to retain a first supply item, wherein the first load cell is configured to detect a first weight of the first tray holder;
a first scale controller comprising a first processor and a first memory, the first scale controller configured to receive the first weight from the first scale and to associate the first weight to a quantity of the first supply item, the first scale controller configured to determine a status of the first supply item based on the first weight of the first tray holder; and
a first remote computing device communicatively coupled to the first scale controller and configured to monitor a status of the first supply item;
wherein the first scale or the first scale controller is operable to perform training of a machine learning model for optimizing supply delivery, the training comprising:
obtaining a dataset of identified supply-related outcomes;
training the machine learning model using the dataset of identified supply-related outcomes thereby obtaining a trained machine learning model; and
storing the trained machine learning model.

2. The system of claim 1, wherein the first scale or the first scale controller is operable to perform further training of a machine learning model for optimizing supply delivery, wherein the further training comprises;
training the machine learning model using a dataset of one or more identified supply-related outcomes, thereby obtaining a further trained machine learning model; and
storing the further trained machine learning model.

3. The system of claim 1, wherein the dataset of identified supply-related outcomes comprise one or more of: supply costs, labor costs, restocking speed, combinations of the foregoing, or other outputs desired to be optimized.

4. The system of claim 1, wherein the machine learning model uses one or more inputs comprising one or more of: geo-location data of workers or supplies, routes, room numbers or identifiers, ASN, shipping/tracking numbers, nurse call-down data, scale controller or sensor data, detour data, or other supply data or building/location-related data.

5. The system of claim 1, wherein the first load cell comprises a strain gauge.

6. The system of claim 1, wherein the first scale controller is coupled to the first scale by at least one of: a wireless connection; a hard wire connection.

7. The system of claim 1, further comprising:
a second scale comprising a second load cell and configured to receive a second tray holder, the second tray holder configured to retain a second supply item, wherein the second load cell is configured to detect a second weight of the second tray holder;
wherein the first scale controller is configured to receive the second weight from the second scale and to associate the second weight to a quantity of the second supply item, the first scale controller configured to determine a status of the second supply item based on the second weight of the second tray holder; and
wherein the first remote computing device is configured to monitor a status of the second supply item.

8. The system of claim 1, wherein the first scale controller is coupled to the first remote computing device by at least one of: a wireless connection; a hard wire connection.

9. A system for monitoring a supply inventory at a location, the system comprising:
a first scale comprising a first load cell and configured to receive a first tray holder, the first tray holder configured to retain a first supply item, wherein the first load cell is configured to detect a first weight of the first tray holder;
a second scale comprising a second load cell and configured to receive a second tray holder, the second tray holder configured to retain a second supply item, wherein the second load cell is configured to detect a second weight of the second tray holder;
a first scale controller, the first scale controller configured to receive the first weight from the first scale and to associate the first weight to a quantity of the first supply item, the first scale controller configured to determine a status of the first supply item based on the first weight of the first tray holder, the first scale controller further configured to receive the second weight from the second scale and to associate the second weight to a quantity of the second supply item, the first scale controller configured to determine a status of the second supply item based on the second weight of the second tray holder;
a first mobile cart configured to carry the first and second supply items, the first mobile cart comprising a first scanner configured to scan the first and second supply items and record a presence of the first and second supply items on the first mobile cart;
a first sensor at the location, the first sensor operable to detect a location of the first and second supply items; and
a first server communicatively coupled to the first scale controller, to the first mobile cart, and the first sensor, and configured to monitor the status of the first supply item, the status of the second supply item, the location of the first and second supply items, and the presence of the first and second supply items on the first mobile cart;
wherein the first scale or the second scale or the first scale controller is operable to perform training of a machine learning model for optimizing supply delivery, the training comprising:
obtaining a dataset of identified supply-related outcomes;
training the machine learning model using the dataset of identified supply-related outcomes thereby obtaining a trained machine learning model, and
storing the trained machine learning model.

10. The system of claim 9, wherein the first scale or the second scale or the first scale controller is operable to perform further training of a machine learning model for optimizing supply delivery, wherein the further training comprises;
training the machine learning model using a dataset of one or more identified supply-related outcomes, thereby obtaining a further trained machine learning model; and
storing the further trained machine learning model.

11. The system of claim 9, wherein the dataset of identified supply-related outcomes comprise one or more of: supply costs, labor costs, restocking speed, combinations of the foregoing, or other outputs desired to be optimized.

12. The system of claim 9, wherein the machine learning model uses one or more inputs comprising one or more of: geo-location data of workers or supplies, routes, room numbers or identifiers, ASN, shipping/tracking numbers, nurse call-down data, scale controller or sensor data, detour data, or other supply data or building/location-related data.

13. The system of claim 9, wherein the first load cell detects the first weight via deformation caused by the first weight.

14. The system of claim 9, wherein the first scale controller is coupled to the first scale by at least one of: a wireless connection; a hard wire connection.

15. The system of claim 9, further comprising:
a third scale comprising a third load cell and configured to receive a third tray holder, the third tray holder configured to retain a third supply item, wherein the third load cell is configured to detect a third weight of the third tray holder;
wherein the first scale controller is further configured to receive the third weight from the third scale and to associate the third weight to a quantity of the third supply item, the first scale controller configured to determine a status of the third supply item based on the third weight of the third tray holder; and
wherein the first server device is further configured to monitor a status of the third supply item.

16. A computer implemented method for training a machine learning model for optimizing supply delivery, the method comprising:
obtaining a dataset of identified supply-related outcomes;
training the machine learning model using the dataset of identified supply-related outcomes thereby obtaining a trained machine learning model, and
storing the trained machine learning model.

17. The method of claim 16, further comprising training a machine learning model for optimizing identified supply-related outcomes, wherein the training comprises;
training the machine learning model using a dataset of one or more identified supply-related outcomes, thereby obtaining a further trained machine learning model; and
storing the further trained machine learning model.

18. The method of claim 16, wherein the one or more identified supply-related outcomes comprise one or more of: supply costs, labor costs, restocking speed, combinations of the foregoing, or other outputs desired to be optimized.

19. The method of claim 16, wherein the machine learning model uses one or more inputs comprising one or more of: geo-location data of workers or supplies, routes, room numbers or identifiers, ASN, shipping/tracking numbers, nurse call-down data, scale controller or sensor data, detour data, or other supply data or building/location-related data.

20. The method of claim 16, further comprising:
receiving a first tray holder in a first scale, the first tray holder configured to retain a first supply item;
detecting, by a first load cell in the first scale, a weight of the first tray holder;
transmitting the weight of the first tray holder to a first scale controller;
associating, by the first scale controller, the weight of the first tray holder to an associated quantity of the first supply item;
determining, by the first scale controller, a status of the first supply item based on the associated quantity and based on the machine learning model; and
transmitting, by the first scale controller, the status of the first supply item to a first remote computing device configured to monitor the status of the first supply item.

21. A method of monitoring supply items, comprising:
receiving a first tray holder in a first scale, the first tray holder configured to retain a first supply item;
detecting, by a first load cell in the first scale, a weight of the first tray holder;
transmitting the weight of the first tray holder to a first scale controller;
associating, by the first scale controller, the weight of the first tray holder to an associated quantity of the first supply item;
determining, by the first scale controller, a status of the first supply item based on the associated quantity and based on the machine learning model; and
transmitting, by the first scale controller, the status of the first supply item to a first remote computing device configured to monitor the status of the first supply item.

22. The method of claim 21, wherein the first load cell comprises a strain gauge.

23. The method of claim 21, further comprising:
receiving a second tray holder in a second scale, the second tray holder configured to retain a second supply item;
detecting, by a second load cell in the second scale, a weight of the second tray holder;
transmitting the weight of the second tray holder to the first scale controller;
associating, by the first scale controller, the weight of the second tray holder to an associated quantity of the second supply item;
determining, by the first scale controller, a status of the second supply item based on the associated quantity; and
transmitting, by the first scale controller, the status of the second supply item to a first remote computing device configured to monitor the status of second first supply item.

24. The method of claim 21, wherein the first scale controller is coupled to the first scale via at least one of: a wireless connection; a hard wire connection.

25. The method of claim 21, wherein the first scale controller is coupled to the first remote computing device via at least one of: a wireless connection; a hard wire connection.

26. The method of claim 21, further comprising transmitting, by the first remote computing device, a notification to a user when the status of the first supply item is low.

27. The method of claim 21, further comprising training a machine learning model for optimizing supply delivery, the training comprising:
obtaining a dataset of identified supply-related outcomes;
training the machine learning model using the dataset of identified supply-related outcomes thereby obtaining a trained machine learning model, and
storing the trained machine learning model.

28. The method of claim 27, further comprising further training a machine learning model for optimizing identified supply-related outcomes, wherein the further training comprises;
training the machine learning model using a dataset of one or more identified supply-related outcomes, thereby obtaining a further trained machine learning model; and
storing the further trained machine learning model.

29. The method of claim 27, wherein the one or more identified supply-related outcomes comprise one or more of: supply costs, labor costs, restocking speed, combinations of the foregoing, or other outputs desired to be optimized.

30. The method of claim 27, wherein the machine learning model uses one or more inputs comprising one or more of: geo-location data of workers or supplies, routes, room numbers or identifiers, ASN, shipping/tracking numbers, nurse call-down data, scale controller or sensor data, detour data, or other supply data or building/location-related data.
